(19) United States
(12) Patent Application Publication
(10) Pub. No.: US 2015/0011473 A1
(43) Pub. Date: Jan. 8, 2015

(54) STABILIZED PTH FORMULATION

(71) Applicant: LUPIN LIMITED, Mumbai, Maharashtra (IN)

(72) Inventors: Vaibhav Dnyaneshwar Deokar, Pune (IN); Anjali Deepak Apte-Deshpande, Pune (IN); Sheetal Arvind Raut, Pune (IN); Balaji Damodaran, Pune (IN); Cyrus Karkaria, Pune (IN)

(21) Appl. No.: 14/373,288

(22) PCT Filed: Jan. 19, 2013
(86) PCT No.: PCT/IB2013/050503

§ 371 Date: Jul. 18, 2014

(30) Foreign Application Priority Data
Jan. 20, 2012 (IN) 53/KOL/2012

(51) Int. Cl. A61K 38/29; A61K 47/12

(52) U.S. Cl. CPC A61K 38/29; A61K 47/12
USPC 514/11.8

(57) ABSTRACT

Stable pharmaceutical formulations comprising human parathyroid hormone are provided. The stabilized aqueous pharmaceutical formulation comprises human parathyroid hormone and a buffer selected from lactate or glutamate. In another embodiment a stabilized aqueous pharmaceutical formulation comprising human parathyroid hormone selected from the group of (1-34), (1-37), (1-38), (1-41), a buffer selected from lactate or glutamate, a stabilizing agent and a parenterally acceptable preservative, wherein the said formulation is sterile and ready for parenteral administration and having pH in the range of 3 to 7 is provided.

### FIELD OF INVENTION

The invention provides aqueous stable pharmaceutical formulations comprising human parathyroid hormone.

### BACKGROUND OF INVENTION

Parathyroid hormone (PTH) is secreted by the chief cells of the parathyroid glands. These glands are also involved in controlling the calcium amount in the blood and bones. They are sensitive to small changes in Ca⁺² concentrations. Initially, the parathyroid hormone is synthesized as a larger preprohormone which is 115 amino acids in length. This preprohomone is later cleaved in rough endoplasmic reticulum and then in Golgi apparatus to form a biologically active hormone, which is an 84 amino acid peptide and the molecular weight is 9425 daltons (Kim et al. 2009 Korean J. Lab. Med. 29, 104-109). The main biological active part of the PTH is the initial 34 amino-terminal amino acids. The carboxyl terminal fragment of the PTH is biologically inactive. Further cleavage of the PTH can occur either in the parathyroid glands or in the blood circulation. The truncated PTH, which is produced by the cleavage from one or both (amino and carboxy) terminal(s) has less or no biological activity. The secretion of PTH is controlled by a negative feedback system. The circulating concentration of Ca⁺² is detected by a unique G-protein-linked calcium receptor (CaR). When the Ca⁺² concentration increases, it stimulates phospholipase C (PLC) and inhibits adenylatedcyclase (AC) which further reduces PTH release and vice versa. It can be concluded that the PTH secretion is inversely proportional to serum Ca⁺² concentrations. When the Ca⁺² concentrations are within the normal limits, both the pathways are balanced and basal secretions of PTH are maintained.

PTH acts on bones to increase the movement of Ca⁺² from bone to blood. It also stimulates osteocytes for bone formation as well as resorption. It enhances reabsoprtion of Ca⁺² in the nephrons, reducing the excretion of Ca⁺² and stimulates calcitriol production which increases intestinal absorption of Ca⁺². In recent studies, for treating osteoporosis, small amount of PTH is injected which helps in bone formation and bone strengthening (Cosman et al. 2002 Osteoporos Int. 13(4), 267-77). PTH increases osteoblast production rate and inhibits its apoptosis which further lead to an increase in skeletal mass and improves bone micro-architecture (Lyritis et al. 2010 Ann. N. Y. Acad. Sci. 1205, 277-283).

PTH is used as an anabolic agent for the treatment of osteoporosis (Black et al. 2003 N Engl J Med. 349, 1207-1215; Jodar-Gimeno 2007 ClinIntery Aging 2, 163-174; Hodsman et al. 2003 J ClinEndocrinolMetab. 88, 5212-5220). Two forms of recombinant human parathyroid hormone (r-hPTH) are widely used. First form is hPTH (1-34) which is 34 residue amino-terminal of parathyroid hormone.hPTH (1-34) has a molecular weight of 4117.8 daltons and its amino acid sequence is shown as: H-Ser-Val-Ser-Glu-Ile-Gln-Leu-Met-His-Asn-Leu-Gly-Lys-His-Leu-Asn-Ser-Met-Glu-Arg-Val-Glu-Trp-Leu-Arg-Lys-Lys-Leu-Gln-Asp-Val-His-Asn-Phe-OH. Recombinant parathyroid hormone highly stimulates the bone formation than resorption (Resimini et al. 2011 Aging ClinExp Res 23, 30-32; Borba et al 2010 Arq Bras EndocrinolMetabol 54(2), 213-9). The second form of r-hPTH is Preotact, which is the intact active 84 amino acid human PTH (1-84).

Proteins achieved through recombinant DNA technology are in a pure form. They are not very stable under normal atmospheric conditions. So it becomes important to make a stable pharmaceutical formulations which delays the degradation of the active principle ingredient (API). Commercial usage of this hormone requires the development of a formulation that will impart storage stability, retains the bioactivity and is easy to prepare.

Formulation of parathyroid hormone is labile due to degradation. It is more labile than the traditional small molecules. It is highly sensitive to oxidation at methionine residues in the positions 8 and 18 giving rise to oxidized PTH species. Furthermore it can get deamidated at asparagine residue in position 16. There is a probability of truncation of polypeptide chain at N-terminal and C-terminals due to breakage of peptide bond. All these reactions can significantly hamper the bioactivity of this protein. Appropriate formulation of PTH will prevent these adverse reactions.

US Pat. Nos U.S. Pat. No. 7,550,434; U.S. Pat. No. 7,144,861 and U.S. Pat. No. 6,770,623 discloses pharmaceutical formulations comprising hPTH (1-34).

PCT application WO 2006/129995 discloses a liquid parathyroid hormone comprising a parathyroid hormone.

### SUMMARY OF THE INVENTION

In an embodiment, the invention is related to stable pharmaceutical formulations comprising a biologically active hPTH and a buffer selected from Lactate buffer or glutamate buffer.

In another embodiment, the invention is related to stable pharmaceutical formulation comprising hPTH and a buffer selected from lactate or glutamate having a pH range of 3.0 to 7.0.

In yet another embodiment, the invention is related to the pharmaceutical formulation further comprising one or more tonicity agents or preservative.

In another embodiment, the parathyroid hormone is selected from the group consisting of hPTH (1-34), hPTH (1-37), hPTH (1-38), hPTH (1-41) and hPTH (1-84).

In an embodiment, the invention is related to stable pharmaceutical formulation comprising a biologically active hPTH (1-34) and a buffer selected from Lactate buffer or glutamate buffer.

The details of one or more embodiments of the invention set forth in the below are illustrative in nature only and not intended to limit to the scope of the invention. Other features, objects and advantages of the inventions will be apparent from the description and claims.

The present invention is related to a stable aqueous pharmaceutical formulation in a pre-filled syringe, vial, cartridge, or pen. In a preferred embodiment invention is related to a stable aqueous pharmaceutical formulation in a vial, cartridge, or pen.

### DETAIL DESCRIPTION OF INVENTION

The invention provides a stable aqueous pharmaceutical formulation comprising hPTH. The pharmaceutical formulation solution is sterile and can be stored for a long period of time. The invention provides for a pharmaceutical formulation in a cartridge comprising a stable hPTH and a buffer selected from lactate or glutamate. The formulation of the invention is sterile and ready for parenteral administration.

In an embodiment of the invention, the biologically active hPTH is selected from the group comprising hPTH (1-34), hPTH (1-37), hPTH (1-38), hPTH (1-41) and hPTH (1-84). The concentration of the hPTH is from 10 μg/ml to 1000 μg/ml, the preferred concentration is 25 μg/ml.

In an embodiment of the invention, lactic acid and sodium lactate constitute lactate buffer and glutamic acid and sodium glutamate constitute glutamate buffer. In an embodiment of the invention, the concentration of the buffer in the solution is 1 mM to 100 mM and the preferred concentration is 10 mM.

In an embodiment of the invention, the buffering system used is acid and salt combination, which is used to maintain the pH of the aqueous solution. In an embodiment the pH range of the formulation of the invention is in the range of 3.0 to 7.0. The preferred pH is 4.0.

In an embodiment of the invention, the stabilizing agent incorporated in the solution is selected from a group of saccharide such as mannitol, glycine, glycerol; chelators selected from the group of EDTA, DTPA or EGTA; amino acid selected from the group of proline, alanine, arginine, asparagines, aspartic acid, cysteine, glutamine, glutamic acid, glycine, histidine, isoleucine, leucine, lysine, methionine, phenylalanine, serine, threonine, tryptophan, tyrosine, and valine; NaCl and the like. The preferred stabilizing agent is mannitol. The concentration of the stabilizing agent varies from about 2 to 20 wt-% of the total solution.

In another embodiment of the invention, the stabilized aqueous composition comprises a parenterally acceptable preservative. Examples of the preservatives are selected from a group of cresols such as metacresol, paracresol, orthocresol; phenol, benzyl alcohol, paraben, thimerosal, benzalkonium chloride, chlorobutanol, benzethonium chloride, chlorobutanol and the like. The preferred preservative is metacresol and the concentration range was about 0.1 to 2 wt % of the total solution.

In another embodiment of the invention, the formulation is a stable hPTH (1-34) solution comprising lactate or glutamate buffer, mannitol as a stabilizing agent and metacresol as a preservative with a long shelf life at temperature ranging from 5° C. to 40° C., preferably 5° C. The formulation has a long shelf life at 5° C.

In yet another embodiment, the invention is related to the method of treating a disease using the stable pharmaceutical formulation of the present invention. The disease may be glucocorticoid-induced osteoporosis in men and women or postmenopausal induced osteoporosis in women or increase in bone mass in men with primary or hypogonadal osteoporosis.

### EXPERIMENTAL SECTION

The examples which follow are illustrative of the invention and are not intended to be limiting.

### Example 1

0.25 mg hPTH (1-34), 45.4 mg mannitol, 0.3 mg m-cresol, 10 mM of lactic acid and sodium lactate were mixed into a solution with 1 ml of distilled water. pH of the solution was adjusted to 4.0 with sodium hydroxide or hydrochloric acid.

TABLE 1
Unit formula for the formulation of hPTH(1-34) of Example 1.

| Components | Formulation | Unit composition | | Range | |
|---|---|---|---|---|---|
| API | hPTH(1-34) | 0.25 | mg | 25-1000 | μg/ml |
| Buffer | Lactate Buffer | 10 | mM | 10-100 | mM |
| Tonicity agent | Mannitol | 45.4 | mg | 2-20 | wt % |
| Preservative | Metacresol | 0.3 | mg | 0.1-2 | wt % |
| pH | 4.0 | | | 3-7 | |

The buffer in this formulation is lactate buffer along with mannitol as a tonicity agent and metacresol as a preservative. According to the results of RP-HPLC and SE-HPLC, it was concluded that the Formulation of example 1 was stable.

### Example 2

0.25 mg hPTH (1-34), 45.4 mg mannitol, 0.3 mg m-cresol, 10 mM of glutamic acid and sodium glutamate were mixed into a solution with 1 ml of distilled water. pH of the solution was adjusted to 4.0 pH with sodium hydroxide or hydrochloric acid.

TABLE 2

| | Formulation |
|---|---|
| API | hPTH(1-34) |
| Buffer | Glutamic acid&Sodium glutamate |
| Tonicity agent | Mannitol |
| Preservative | Metacresol |
| pH | 4.0 |

The above formulations of Example 1 and Example 2 were prepared by gel filtration chromatography (GFC) of drug substance which was in acetate buffer. GFC was carried out for the buffer exchange to get the desired formulation where protein concentration after buffer exchange was ˜0.6 mg/ml in respective formulation. It was further diluted with the same buffer to achieve the final protein concentration of 0.25 mg/ml. These formulations were filled aseptically into cartridges of volume 3 ml and were maintained at 5° C. and 40° C. to check the stability of the protein. The stability of the protein at various time points (0, 3, 9 and 12 months) was determined by checking protein profile by RP-HPLC, SE-HPLC. The pH, osmolality and bioactivity of hPTH (1-34) of the formulations were determined after 12 months. Acetate estimation was carried out for all the formulations to ensure appropriate buffer exchange during GFC step. The potency of hPTH (1-34) was also calculated after 3 months and 12 months. Initially the potency of example 1 was 0.92×10⁴ IU/mg, after 3 months the potency 5° C. was 1.27×10⁴ IU/mg and after 12 months the potency at 5° C. was 1.14×10⁴ IU/mg. Further, the potency of example 1 after 3 months at 40° C. was 0.99×10⁴ IU/mg. For example 2, the initial potency was 1.2×10⁴ IU/mg, after 3 months the potency at 5° C. was 0.85×10⁴ IU/mg and after 12 months the potency at 5° C. was 1.21×10⁴ IU/mg. Further the potency of example 2 after 3 months at 40° C. was 0.85×10⁴ IU/mg.

The formulation of Example 1 and Example 2 were found to be stable at 5° C. for a period of more than one year.

The formulations of Example-1 and Example-2 were found to be stable at 40° C. up to 4 weeks.

### Example 3

TABLE 3

| | Formulation |
|---|---|
| API | hPTH(1-34) |
| Buffer | Lactic acidSodium lactate |
| Tonicity agent | Mannitol |
| Preservative | Metacresol |
| pH | 4.0 |

The formulation of this example comprises mannitol as a stabilizing agent; lactic acid and sodium lactate as buffering agents, which maintains the pH at 4.0 and metacresol as a preservative. The concentration of mannitol is 45.4 mg/ml.

### Example 4

TABLE 4

| | Formulation |
|---|---|
| API | hPTH(1-34) |
| Buffer | Lactic acidSodium lactate |
| Tonicity agent | Glycerol |
| Preservative | Metacresol |
| pH | 4.0 |

The formulation of this example comprises glycerol as a stabilizing agent; lactic acid and sodium lactate as buffering agents, which maintains the pH at 4.0 and metacresol as a preservative. The concentration of glycerol is 23.02 mg/ml.

### Example 5

TABLE 5

| | Formulation |
|---|---|
| API | hPTH(1-34) |
| Buffer | Lactic acid - Sodium lactate |
| Tonicity agent | Glycine |
| Preservative | Metacresol |
| pH | 4.0 |

The formulation of this example comprises glycine as a stabilizing agent; lactic acid and sodium lactate as buffering agents, which maintains the pH at 4.0 and metacresol as a preservative. The concentration of glycine for this example is 18.76 mg/ml.

### Example 6

TABLE 6

| | Formulation |
|---|---|
| API | hPTH(1-34) |
| Buffer | Lactic acid, Sodium lactate |
| Tonicity agent | NaCl (7.3 mg/ml) |
| Preservative | Metacresol |
| pH | 4.0 |

The formulation of this example comprises sodium chloride as a stabilizing agent; lactic acid and sodium lactate as buffering agents, which maintains the pH at 4.0 and metacresol as a preservative. The concentration of sodium chloride for this example is 7.3 mg/ml.

All patents, patent applications and publications cited in this application are hereby incorporated by reference in their entirety for all purposes to the same extent as if each individual patent, patent application or publication were so individually denoted.

Although certain embodiments and examples have been described in detail above, those having ordinary skill in the art will clearly understand that many modifications are possible in the embodiments and examples without departing from the teachings thereof.

1. A stabilized aqueous pharmaceutical formulation comprising human parathyroid hormone and a buffer selected from lactate or glutamate.

2. A stabilized aqueous pharmaceutical formulation comprising human parathyroid hormone selected from the group of (1-34), (1-37), (1-38), (1-41), a buffer selected from lactate or glutamate, a stabilizing agent and a parenterally acceptable preservative, wherein the said formulation is sterile and ready for parenteral administration having pH in the range of 3 to 7.

3. The pharmaceutical formulation of claim 2, wherein the human parathyroid hormone is hPTH (1-34).

4. The pharmaceutical formulation of claim 2, wherein the stabilizing agent is selected from the group consisting of mannitol, glycine, glycerol, EDTA, DTPA or EGTA, proline, alanine, arginine, asparagines, aspartic acid, cysteine, glutamine, glutamic acid, glycine, histidine, isoleucine, leucine, lysine, methionine, phenylalanine, serine, threonine, tryptophan, tyrosine, valine and NaCl.

5. The pharmaceutical formulation of claim 4, wherein the stabilizing agent is mannitol.

6. The pharmaceutical formulation of claim 2, wherein the preservative is metacresol.

7. A stabilized aqueous pharmaceutical formulation comprising human parathyroid hormone (1-34); a buffer selected from lactate or glutamate; mannitol as a stabilizing agent and metacresol as a parenterally acceptable preservative; wherein the said formulation is sterile and ready for parenteral administration.

8. The pharmaceutical formulation of claim 7, comprising about 10 μg/ml to 1000 μg/ml of hPTH (1-34), about 1 mM to 100 mM of lactate buffer, about 2 to 20 wt-% of mannitol, about 0.1 to 2 wt % of metacresol having pH in the range of pH 3.0 to 7.0.

9. The pharmaceutical formulation of claim 7 comprising about 10 μg/ml to 1000 μg/ml of hPTH (1-34), about 1 mM to 100 mM of glutamate buffer, about 2 to 20 wt-% of mannitol, about 0.1 to 2 wt % of metacresol having pH in the range of pH 3.0 to 7.0.

10. A method for treating osteoporosis comprising administering the pharmaceutical formulation of claim 1.
